(19) United States
(12) Patent Application Publication
(10) Pub. No.: US 2024/0216092 A1
(43) Pub. Date: Jul. 4, 2024

(54) ELONGATE MEMBER WITH COUPLER TO PROVIDE RADIAL TRANSITION OF TRANSLATING MEMBER

(71) Applicant: Auris Health, Inc., Redwood City, CA (US)

(72) Inventors: Taylor E. Furtado, Campbell, CA (US); Sean N. Finson, Santa Clara, CA (US)

(21) Appl. No.: 18/536,619

(22) Filed: Dec. 12, 2023

Related U.S. Application Data
(60) Provisional application No. 63/436,193, filed on Dec. 30, 2022.

(51) Int. Cl. A61B 34/00; A61B 1/005; A61B 1/307; A61B 17/221; A61B 34/37

(52) U.S. Cl. CPC A61B 34/71; A61B 1/0057; A61B 1/307; A61B 17/221; A61B 34/37; A61B 2017/2215

(57) ABSTRACT

An apparatus includes a proximal elongate portion, a distal elongate portion, a coupler, a proximal tendon assembly, and a distal tendon assembly. The proximal and distal elongate portions share a central longitudinal axis. The coupler is longitudinally interposed between the proximal elongate portion and the distal elongate portion. The coupler includes a first channel. The proximal tendon assembly extends through the proximal elongate portion. The proximal tendon assembly includes a proximal portion of a tendon positioned at a first radial distance from the central longitudinal axis. The distal tendon assembly extends through the distal elongate portion. The distal tendon assembly includes a distal portion of the tendon positioned at a second radial distance from the central longitudinal axis. The tendon passes through the channel of the coupler, which accommodates repositioning of the tendon from the first radial distance to the second radial distance.

### PRIORITY

This application claims the benefit of U.S. Pat. App. No. 63/436,193, entitled “Elongate Member with Coupler to Provide Radial Transition of Translating Member,” filed Dec. 30, 2022, the disclosure of which is incorporated by reference herein.

### BACKGROUND

A variety of surgical instruments include an end effector for use in medical treatments and procedures conducted by a medical professional operator, including applications in robotically assisted surgeries. In the case of robotically assisted surgery, the surgeon may operate a master controller to remotely control the motion of such surgical instruments at a surgical site. The controller may be separated from the patient by a significant distance (e.g., across the operating room, in a different room, or in a completely different building than the patient); or quite near the patient in the operating room. The controller may include one or more hand input devices (e.g., joysticks, exoskeletal gloves, master manipulators, etc.), which are coupled by a servo mechanism to the surgical instrument. In one example, a servo motor moves a manipulator supporting the surgical instrument based on the surgeon's manipulation of the hand input devices. During the surgery, the surgeon may employ, via a robotic surgical system, a variety of surgical instruments including an ultrasonic blade, a surgical stapler, a tissue grasper, a needle driver, an electrosurgical cautery probes, etc. Each of these structures performs functions for the surgeon, for example, cutting tissue, coagulating tissue, manipulating a needle, grasping a blood vessel, dissecting tissue, or cauterizing tissue. A robotically-controlled instrument may be introduced into the patient via an incision, via a naturally occurring orifice, or otherwise.

While several robotic surgical systems and associated components have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a top plan view of an example of a robotic surgical system being used in a urological procedure.

FIG. 2 depicts a schematic view of different components of the robotic surgical system of FIG. 1.

FIG. 3 depicts enlarged views of other components of the robotic surgical system of FIG. 1, including a distal portion of a ureteroscope.

FIG. 4 depicts a schematic view of an example of an articulating elongate member that may be used with the robotic surgical system of FIG. 1.

FIG. 5 depicts a cross-sectional end view of the elongate member of FIG. 4, taken along line 5-5 of FIG. 4.

FIG. 6 depicts a cross-sectional end view of the elongate member of FIG. 4, taken along line 6-6 of FIG. 4.

FIG. 7 depicts a perspective view of a coupler of the elongate member of FIG. 4.

FIG. 8 depicts another perspective view of the coupler of FIG. 7.

FIG. 9 depicts a side view of a portion of the elongate member of FIG. 4, showing a transition of tendon assemblies along the coupler of FIG. 7.

FIG. 10 depicts a cross-sectional side view of the elongate member of FIG. 4, taken along line 10-10 of FIG. 4.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. It will be further appreciated that, for convenience and clarity, spatial terms such as “side,” “upwardly,” and “downwardly” also are used herein for reference to relative positions and directions. Such terms are used below with reference to views as illustrated for clarity and are not intended to limit the invention described herein.

Aspects of the present examples described herein may be integrated into a robotically-enabled medical system, including as a robotic surgical system, capable of performing a variety of medical procedures, including both minimally invasive, such as laparoscopy, and non-invasive, such as endoscopy, procedures. Among endoscopy procedures, the robotically-enabled medical system may be capable of performing bronchoscopy, ureteroscopy, gastroscopy, etc.

In addition to performing the breadth of procedures, the robotically-enabled medical system may provide additional benefits, such as enhanced imaging and guidance to assist the medical professional. Additionally, the robotically-enabled medical system may provide the medical professional with the ability to perform the procedure from an ergonomic position without the need for awkward arm motions and positions. Still further, the robotically-enabled medical system may provide the medical professional with the ability to perform the procedure with improved ease of use such that one or more of the instruments of the robotically-enabled medical system may be controlled by a single operator.

### I. EXAMPLE OF ROBOTICALLY-ENABLED MEDICAL SYSTEM

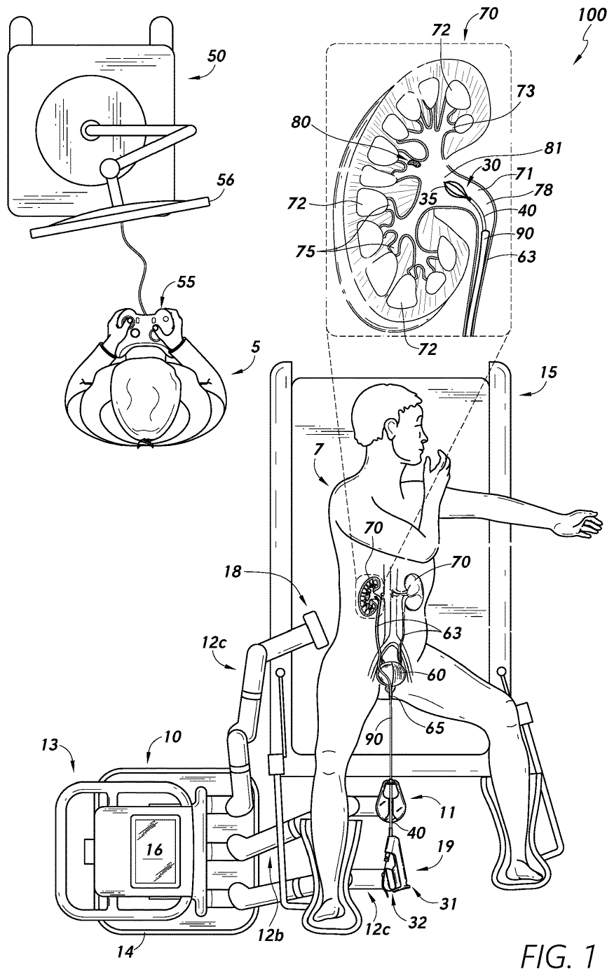

FIG. 1 shows an example medical system (100) for performing various medical procedures in accordance with aspects of the present disclosure. The medical system (100) may be used for, for example, endoscopic (e.g., ureteroscopic) procedures. Certain ureteroscopic procedures involve the treatment/removal of kidney stones. Although the system (100) of FIG. 1 is presented in the context of a ureteroscopic procedure, it should be understood that the principles disclosed herein may be implemented in any type of endoscopic (e.g., bronchial, gastrointestinal, etc.) and/or percutaneous procedure.

The medical system (100) of the present includes a robotic system (10) (e.g., mobile robotic cart) that is configured to engage with and/or control one or more medical instruments (e.g., ureteroscope (40), basketing system (30), etc.) via one or more robotic arms (12) to perform a direct-entry procedure on a patient (7). In some versions, the robotic system (10) and/or control system (50) is/are configured to receive images and/or image data from the scope (40) representing internal anatomy of the patient (7), namely the urinary system with respect to the particular depiction of FIG. 1, and/or display images based thereon.

It should be understood that the direct-entry instrument(s) operated through systems (10, 50) may include any type of medical instrument or combination of instruments, including an endoscope (such as a ureteroscope (40)), catheter (such as a steerable or non-steerable catheter), nephroscopes, laparoscope, basketing systems (30), and/or other type of medical instrument(s). The various scope-type instruments disclosed herein, such as the scope (40) of the system (100), may be configured to navigate within the human anatomy, such as within a natural orifice or lumen of the human anatomy. The terms “scope” and “endoscope” are used herein according to their broad and ordinary meanings; and may refer to any type of elongate medical instrument having image generating, viewing, and/or capturing functionality and configured to be introduced into any type of organ, cavity, lumen, chamber, or space of a body. A scope may include, for example, a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), borescope, and so on. Scopes/endoscopes, in some instances, may comprise a rigid or flexible tube, and may be dimensioned to be passed within an outer sheath, catheter, introducer, or other lumen-type device, or may be used without such devices.

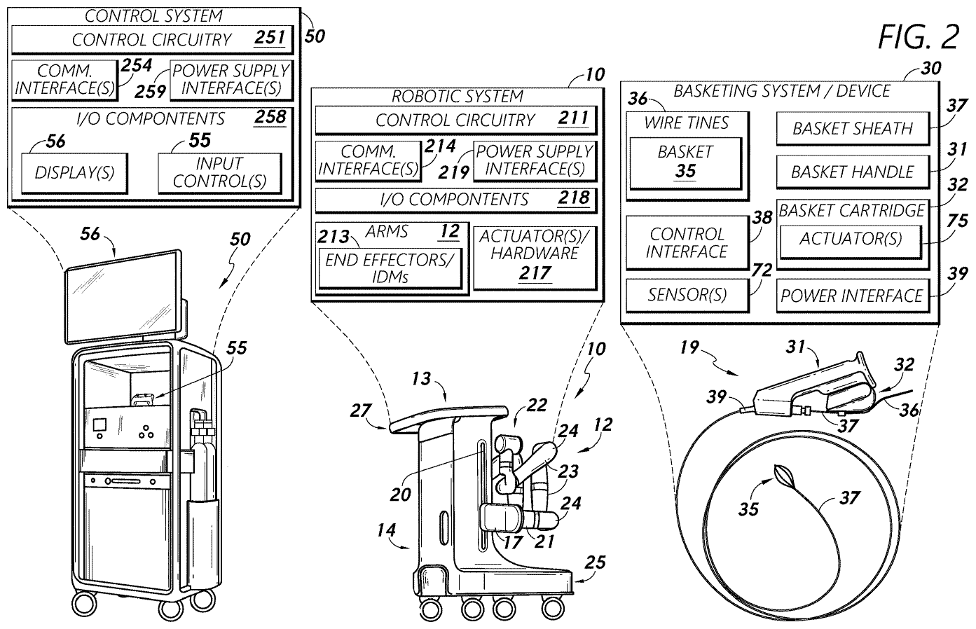

The medical system (100) of the present example further includes a control system (50), a table (15), and an electromagnetic (EM) field generator (18). Table (15) is configured to hold the patient (7). EM field generator (18) may be held by one or more of the robotic arms (12) of the robotic system (10) or may be a stand-alone device. As shown in FIGS. 1-2, control system (50) of the present example includes various input/output (I/O) components (258) configured to assist the physician (5) or others in performing a medical procedure. For example, the I/O components (258) may be configured to allow for user input to control/navigate the scope (40) and/or basketing system (30) within the patient (7). I/O components (258) of the present example include a controller (55) that is configured to receive user input from the operator; and a display (56) that is configured to present certain information to assist the operator. Controller (55) may take any suitable form, including but not limited to one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, etc.

As also shown in FIG. 2, control system (50) of the present example includes a communication interface (254) that is operable to provide a communicative interface between control system (50) and robotic system (10), basketing system (30), scope (40), and/or other components. Communications via communication interface (254) may include data, commands, electrical power, and/or other forms of communication. Communication interface (254) may also be configured to provide communication via wire, wirelessly, and/or other modalities. Control system (50) also includes a power supply interface (259), which may receive power to drive control system (50) via wire, battery, and/or any other suitable kind of power source. A control circuitry (251) of control system (50) may provide signal processing and execute control algorithms to achieve the functionality of medical system (100) as described herein.

The control system (50) may also communicate with the robotic system (10) to receive position data therefrom relating to the position of the distal end of the scope (40), access sheath (90), or basketing device (30). Such positional data relating to the position of the scope (40), access sheath (90), or basketing device (30) may be derived using one or more electromagnetic sensors associated with the respective components. Moreover, in some versions, the control system (50) may communicate with the table (15) to position the table (15) in a particular orientation or otherwise control the table (15). The control system (50) may also communicate with the EM field generator (18) to control generation of an EM field in an area around the patient (7).

As noted above and as shown in FIGS. 1-2, robotic system (10) includes robotic arms (12) that are configured to engage with and/or control scope (40) and/or the basketing system (30) to perform one or more aspects of a procedure. It should be understood that robotic arms (12) may be coupled to different instruments than what is shown in FIG. 1; and in some scenarios, one or more of the robotic arms (12) may not be utilized or coupled to a medical instrument. Each robotic arm (12) includes multiple arm segments (23) coupled to joints (24), which may provide multiple degrees of movement/freedom. In the example of FIG. 1, the robotic system (10) is positioned proximate to the patient's legs and the robotic arms (12) are actuated to engage with and position the scope (40) for access into an access opening, such as the urethra (65) of the patient (7). When the robotic system (10) is properly positioned, the scope (40) may be inserted into the patient (7) robotically using the robotic arms (12), manually by the physician (5), or a combination thereof. A scope-driver instrument coupling (11) (e.g., instrument device manipulator (IDM)) may be attached to the distal portion of one of the arms (12b) to facilitate robotic control/advancement of the scope (40). Another (12c) of the arms may include an instrument coupling/manipulator (19) that is configured to facilitate advancement and operation of the basketing device (30). The scope (40) may include one or more working channels through which additional tools, such as lithotripters, basketing devices, forceps, etc., may be introduced into the treatment site.

The robotic system (10) may be coupled to any component of the medical system (100), such as the control system (50), the table (15), the EM field generator (18), the scope (40), the basketing system (30), and/or any type of percutaneous-access instrument (e.g., needle, catheter, nephroscope, etc.). As noted above, robotic system (10) may be communicatively coupled with control system (50) via communication interfaces (214, 254). Robotic system (10) also includes a power supply interface (219), which may receive power to drive robotic system (10) via wire, battery, and/or any other suitable kind of power source. In addition, robotic system (10) of the present example includes various input/output (I/O) components (218) configured to assist the physician (5) or others in performing a medical procedure. Such I/O components (218) may include any of the various kinds of I/O components (258) described herein in the context of control system (50). In addition, or in the alternative, I/O components (218) of robotic system (10) may take any suitable form (or may be omitted altogether).

Robotic system (10) of the present example generally includes a column (14), a base (25), and a console (13) at the top of the column (14). The column (14) may include one or more arm supports (17) (also referred to as a “carriage”) for supporting the deployment of the one or more robotic arms (12) (three shown in FIG. 2). The arm support (17) may include individually-configurable arm mounts that rotate along a perpendicular axis to adjust the base of the robotic arms (12) for desired positioning relative to the patient. In some versions, the arm support (17) may be connected to the column (14) through slots (20) that are positioned on opposite sides of the column (14) to guide vertical translation of the arm support (17) along column (14). The robotic arms (12) of the present example generally comprise robotic arm bases (21) and end effectors (22), separated by a series of linking arm segments (23) that are connected by a series of joints (24), each joint comprising one or more independent actuators (217). Each actuator (217) may comprise an independently-controllable motor. I/O components (218) may be positioned at the upper end of column (14). Console (13) also includes a handle (27) to assist with maneuvering and stabilizing robotic system (10).

The end effector (213) of each of the robotic arms (12) may include an instrument device manipulator (IDM), which may be attached using a mechanism changer interface (MCI). In some versions, the IDM (213) may be removed and replaced with a different type of IDM (213), for example, a first type (11) of IDM (213) may manipulate a scope (40), while a second type (19) of IDM (213) may manipulate a basketing system (30). Another type of IDM (213) may be configured to hold an electromagnetic field generator (18). An MCI may provide power and control interfaces (e.g., connectors to transfer pneumatic pressure, electrical power, electrical signals, and/or optical signals from the robotic arm (12) to the IDM (213). The IDMs (213) may be configured to manipulate medical instruments (e.g., surgical tools/instruments), such as the scope (40), using techniques including, for example, direct drives, harmonic drives, geared drives, belts and pulleys, magnetic drives, and the like.

The system (100) may include certain control circuitry configured to perform certain of the functionality described herein, including the control circuitry (211) of the robotic system (10) and the control circuitry (251) of the control system (50). That is, the control circuitry of the system (100) may be part of the robotic system (10), the control system (50), or some combination thereof. The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in versions in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The control circuitry (211, 251) may comprise computer-readable media storing, and/or configured to store, hard-coded and/or operational instructions corresponding to at least some of the steps and/or functions illustrated in one or more of the present figures and/or described herein. Such computer-readable media may be included in an article of manufacture in some instances. The control circuitry (211, 251) may be entirely locally maintained/disposed or may be remotely located at least in part (e.g., communicatively coupled indirectly via a local area network and/or a wide area network).

In some versions, for example, the physician (5) may provide input to the control system (50) and/or robotic system (10); and in response to such input, control signals may be sent to the robotic system (10) to manipulate the scope (40) and/or catheter basketing system (30). The control system (50) may include one or more display devices (56) to provide various information regarding a procedure. For example, the display(s) (56) may provide information regarding the scope (40) and/or basketing system (30). The control system (50) may receive real-time images that are captured by the scope (40) and display the real-time images via the display(s) (56).

As shown in FIG. 2, the basketing device (30) of the present example includes a basket (35) formed of one or more wire tines (36) disposed within a basketing sheath (37) over a length thereof, where the tines project from a distal end of the sheath (37) to form the basket (35). The tines (36) further extend from the proximal end of the sheath (37) and are slidable within the basketing sheath (37). The tines (36) and the sheath (37) may be coupled to respective actuators (75) of a basket cartridge component (32). The basket cartridge (32) may be physically and/or communicatively coupled to a handle portion/component (31) of the basketing system (30). The handle component (31) may be configured to be used to assist in basketing control either manually or through robotic control. The basketing system (30) may be powered through a power interface (39) and/or controlled through a control interface (38), each or both of which may interface with a robotic arm/component of the robotic system (10). The basketing system (30) may further comprise one or more sensors (72), such as pressure and/or other force-reading sensors, which may be configured to generate signals indicating forces experienced at/by one or more of the actuators (75) and/or other couplings of the basketing system (30).

In an example of a use case, if the patient (7) has a kidney stone (80) located in the kidney (70), the physician may execute a procedure to remove the stone (80) through the urinary tract (65, 60, 63). In particular, and as shown in FIG. 1, the physician may operate medical system (100) to achieve direct entry of the scope (40) into the urinary tract (65, 60, 63) of the patient (7) via the urethra (65). The physician (5) may interact with the control system (50) and/or the robotic system (10) to cause/control the robotic system (10) to advance and navigate the scope (40) from the urethra (65), through the bladder (60), up the ureter (63), and into the renal pelvis (71) and/or calyx network of the kidney (70) where the stone (80) is located. The physician (5) may further interact with the control system (50) and/or the robotic system (10) to cause/control the advancement of a basketing device (30) through a working channel of the scope (40), where the basketing device (30) is configured to facilitate capture and removal of a kidney stone. The control system (50) may provide information via the display(s) (56) that is associated with the medical instrument (40), such as real-time endoscopic images captured therewith, and/or other instruments of the system (100), to assist the physician (5) in navigating/controlling such instrumentation.

In the present example, a ureteral access sheath (90) is disposed within the urinary tract (65, 60, 63) to an area near the kidney (70). The scope (40) may be passed through the ureteral access sheath (90) to gain access to the internal anatomy of the kidney (70), as shown. Once at the site of the kidney stone (80) (e.g., within a target calyx (73) of the kidney (70) through which the stone (80) is accessible), the scope (40) may be used to channel/direct the basketing device (30) to the target location. Once the stone (80) has been captured in the distal basket portion (35) of the basketing device (30), the utilized ureteral access path may be used to extract the kidney stone (80) from the patient (7).

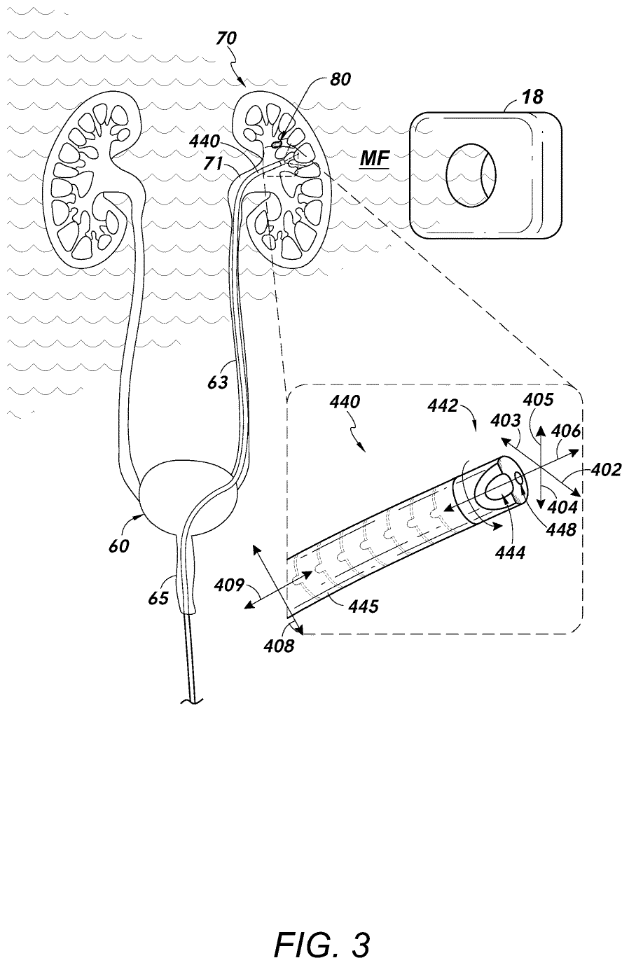

FIG. 3 shows an example of a scope (440) that may be used as scope (40) described above. Scope (440) of this example includes a working channel (444) for deploying medical instruments (e.g., lithotripters, basketing system (30), forceps, etc.), irrigation, and/or aspiration to an operative region at a distal end of the scope (440). The scope (440) may be articulated, such as with respect to at least a distal portion of the scope (440), so that the scope (440) can be steered within the human anatomy. In some versions, the scope (440) is configured to be articulated with, for example, five degrees of freedom, including XYZ coordinate movement, as well as pitch and yaw. In some versions, the scope (440) provides six degrees of freedom, including X, Y, and Z ordinate positions, as well as pitch, roll, and yaw. Position sensor(s) of the scope (440) may likewise have similar degrees of freedom with respect to the position information they produce/provide. As shown in FIG. 3, the tip (442) of the scope (440) may be oriented with zero deflection relative to a longitudinal axis (406) thereof (also referred to as a “roll axis”).

In the present example, the scope (440) can accommodate wires and/or optical fibers to transfer signals to/from an optical assembly and a distal end (442) of the scope (440), which can include an imaging device (448), such as an optical camera. The imaging device (448) may be used to capture images of an internal anatomical space, such as a target calyx/papilla of the kidney (70). The scope (440) may further be configured to accommodate optical fibers to carry light from proximately-located light sources, such as light-emitting diodes, to the distal end (442) of the scope (440). The distal end (442) of the scope (440) may include ports for light sources to illuminate an anatomical space when using the imaging device (448). The imaging device (448) may comprise an optical fiber, fiber array, and/or lens; or a light-emitting diode at distal end (442). The optical components of imaging device (448) move along with the distal end (442) of the scope (440), such that movement of the distal end (442) of the scope (440) results in changes to the images captured by the imaging device(s) 448.

To capture images at different orientations of the tip (442), robotic system (10) may be configured to deflect the tip (442) on a positive yaw axis (402), negative yaw axis (403), positive pitch axis (404), negative pitch axis (405), or roll axis (406). The tip (442) or body (445) of the scope (442) may be elongated or translated in the longitudinal axis (406), x-axis (408), or y-axis (409). The scope (440) may include a reference structure (not shown) to calibrate the position of the scope (440). For example, robotic system (10) and/or control system (50) may measure deflection of the scope (440) relative to the reference structure. The reference structure may be located, for example, on a proximal end of the endoscope (440) and may include a key, slot, or flange.

A robotic arm (12) of robotic system (10) may be configured/configurable to manipulate the scope (440) as described above. Such manipulation may be performed by actuating one or more elongate members such as one or more pull wires (e.g., pull or push wires), cables, fibers, and/or flexible shafts. For example, robotic arms (12) may be configured to actuate multiple pull wires (not shown) coupled to the scope (440) to deflect the tip (442) of the scope (440). Pull wires may include any suitable or desirable materials, such as metallic and non-metallic materials such as stainless steel, aramid fiber, tungsten, carbon fiber, and the like. In some versions, the scope (440) is configured to exhibit nonlinear behavior in response to forces applied by the elongate movement members. The nonlinear behavior may be based on stiffness and compressibility of the scope (440), as well as variability in slack or stiffness between different elongate movement members.

In some versions, the scope (440) includes at least one sensor that is configured to generate and/or send sensor position data to another device. The sensor position data can indicate a position and/or orientation of the scope (440) (e.g., the distal end (442) thereof) and/or may be used to determine/infer a position/orientation of the scope (440). For example, a sensor (sometimes referred to as a “position sensor”) may include an electromagnetic (EM) sensor with a coil of conductive material or other form of an antenna. In some versions, the position sensor is positioned on the distal end (442) of scope (440), while in other embodiments the sensor is positioned at another location on scope (440).

As shown in FIG. 3, EM field generator (18) is configured to broadcast an alternating EM field 90 that is detected by the EM position sensor of scope (440). The alternating magnetic field (MF) induces small currents in coils of the EM position sensor, which may be analyzed to determine a distance and/or angle/orientation between the EM position sensor and the EM field generator (18). It should be understood that scope (440) may include other types of sensors, such as a shape sensing fiber, accelerometer(s), gyroscope(s), satellite-based positioning sensor(s) (e.g., global positioning system (GPS) sensors), radio-frequency transceiver(s), and so on. In the present example, the EM position sensor of scope (440) provides sensor data to control system (50), which is then used to determine a position and/or an orientation of scope (440).

In some variations, any of the features and aspects described above may be constructed and operable in accordance with at least some of the teachings of U.S. Pat. No. 11,737,663, entitled “Target Anatomical Feature Localization,” issued Aug. 29, 2023, the disclosure of which is incorporated by reference herein, in its entirety; U.S. Pub. No. 2021/0369384, entitled “Stuck Instrument Management,” published Dec. 2, 2021, the disclosure of which is incorporated by reference herein, in its entirety; U.S. Pub. No. 2021/0401527, entitled “Robotic Medical Systems Including User Interfaces with Graphical Representations of User Input Devices,” published Dec. 30, 2021, the disclosure of which is incorporated by reference herein, in its entirety; and/or U.S. Pub. No. 2022/0096183, entitled “Haptic Feedback for Aligning Robotic Arms,” published Mar. 31, 2022, the disclosure of which is incorporated by reference herein, in its entirety.

### II. EXAMPLE OF COUPLER TO PROVIDE RADIAL TRANSITION OF TENDON ASSEMBLIES

As noted above, robotic system (10) may include one or more articulating elongate instruments, such as scope (40), access sheath (90), scope (440), catheters, and/or other kinds of elongate instruments. Some such articulating elongate instruments may include one or more translating features that drive articulation. The inclusion of such translating articulation drive features may tend to be associated with certain design constraints or requirements. For instance, it may be desirable to provide a degree of force isolation relative to the translating articulation drive features along certain longitudinal regions (e.g., a proximal region) of the elongate instrument, such that actuation of the drive feature only causes articulation of another certain longitudinal region (e.g., a distal region) of the elongate instrument. It may also be desirable to minimize friction with respect to translating articulation drive features. Such friction reduction may be particularly challenging in scenarios where the translating articulation drive features must traverse a plurality of elements that move relative to each other (e.g., an array of beads or vertebrae along an articulation joint).

In articulating elongate instruments that include one or more internal working channels that receive other instruments, and that have a maximum cross-sectional area constraint, translating articulation drive features may warrant compromises between achieving an internal working channel of a suitable size and an overall outer diameter of a suitable size. In elongate instruments having additional features in the distal portion of the instrument (e.g., one or more cameras and/or illumination features of scope (40), etc.), it may be desirable for translating articulation drive features to be positioned accommodate structures associated with such additional distal features; yet not be necessary for translating articulation drive features to be positioned accommodate those additional distal features along a proximal portion of the elongate instrument.

It may also be desirable for an elongate instrument to have a proximal portion with an architecture that is substantially different from an architecture of a distal portion of the elongate instrument; or even a series of three or more longitudinally staggered portions each having their own unique architecture. Such differing architectures may include those providing different degrees of flexibility, different kinds of articulation (e.g., single-plane articulation vs. two-plane articulation), a dichotomy of articulating vs. non-articulating, or other kinds of differing architecture. Some such elongate instruments may warrant having translating articulation drive features that traverse the full length of the elongate instrument, such that the translating articulation drive features must pass through these longitudinally varying architectures. This may compound the potential design challenges noted above in the context of translating articulation drive features.

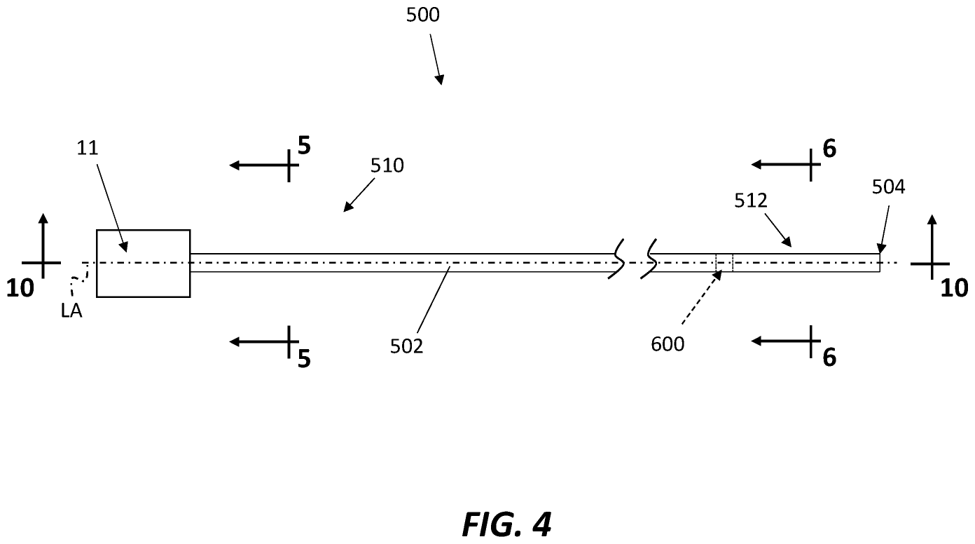

The following describes an example of an articulating elongate member having translating articulation drive features that may readily traverse longitudinally varying architectures along the length of the elongate member, with minimized friction, and while readily accommodating other structural features in the distal portion of the elongate member without adversely affecting the outer diameter of the elongate member. In particular, FIG. 4 shows an example of an articulating elongate member (500) that may be used with robotic surgical system (10). By way of example only, elongate member (500) may represent a variation of scope (40), access sheath (90), or scope (440). Alternatively, elongate member (500) may take the form of a catheter and/or any other suitable kind of elongate instrument. Elongate member (500) of the present example includes a proximal portion (510), a distal portion (512), and a coupler (600) joining portions (510, 512) together. In the present example, distal portion (512) is operable to articulate, such that distal end (504) of elongate member (500) may be deflected laterally away from and toward a central longitudinal axis (LA) (e.g., defined by proximal portion (510)). Also in the present example, proximal potion (510) is flexible yet not configured to articulate. In some variations, elongate member (500) is operable to articulate at one or more different regions along the length of elongate member (500). For instance, distal portion (512) may include one or more articulation sections and/or proximal portion (510) may include one or more articulation sections.

Proximal portion (510) is coupled with instrument coupling (11) of robotic surgical system (10), such that robotic surgical system (10) is operable to drive elongate member (500) via instrument coupling (11). By way of example only, robotic surgical system (10) may be operable to drive translation along the central longitudinal axis (LA), rotation (e.g., spinning about the central longitudinal axis (LA)), articulation, and/or other forms of movement of/by elongate member (500).

Distal end (504) of the present example may include one or more openings through which one or more additional instruments may exit into a surgical space or other anatomical region within a patient. Distal end (504) may also include one or more imaging devices, such as an imaging device (448), that may take the form of one or more cameras, one or more optical fibers with corresponding lenses, etc. Distal end (504) may also include one or more illuminating elements, such as one or more integral light-emitting diodes, one or more lenses optically coupled with corresponding optical fibers, etc. In some versions, distal end (504) includes an end effector that is operable to perform one or more operations on tissue, such as grasping, cutting, suturing, sealing (e.g., via RF energy or ultrasonic energy), stapling, etc.

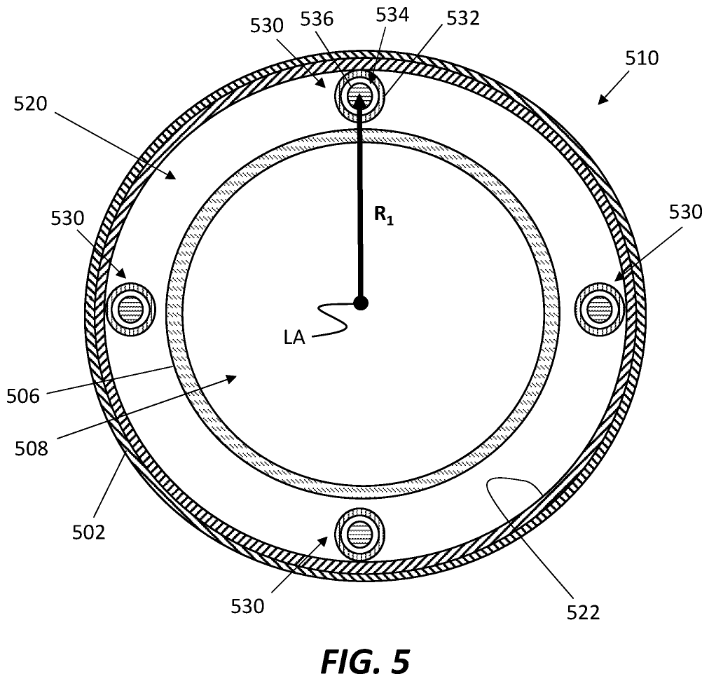

As shown in FIG. 5, proximal portion (510) includes an outer shaft (502) and an inner shaft (522). Shafts (502, 522) may have any suitable configuration. By way of example only, either shaft (502, 522) may include a flexible laser-cut steel hypotube, a braided structure, or any other suitable kind of structure. In some versions, outer shaft (502) provides primary structural support, while inner shaft (522) serves as a liner (e.g., a low friction coating such as polytetrafluoroethylene, etc.). In some other versions, inner shaft (522) provides primary structural support, while outer shaft (502) serves as a liner (e.g., a low friction coating such as polytetrafluoroethylene, etc.). As yet another variation, either shaft (502, 522) may comprise a reflow material such as polyether block amide (PEBA) and/or any other suitable kind(s) of material(s). Some other versions may provide just one single shaft (502, 522), such that the other shaft (502, 522) is omitted.

Inner shaft (522) defines an inner lumen (520). Inner lumen (520) is configured to receive other components. By way of example only, inner lumen may receive coil pipes, Bowden tubes, electrical wires, etc. In the present example, inner lumen receives a working channel (506), which may comprise a braided shaft and/or any other suitable components. Working channel (506) defines a lumen (508). In some versions, lumen (508) slidably receives other instruments. By way of example only, basket (35) and basketing sheath (37) of basketing device (30) may be advanced distally via lumen (508) of working channel (506) in inner lumen (520). By way of further example only, a laser fiber or other instrument may be disposed in lumen (508) of working channel (506). Alternatively, fluids (e.g., liquid, suction, etc.) may be communicated via lumen (508) of working channel (506). Inner lumen (520) and working channel (506) may extend all the way to distal end (504), where inner lumen (520) may terminate in a distal opening allowing an instrument that is disposed in working channel (506) to exit distally from elongate member (500). While not shown, other features such as electrical wires, optical fibers, flex circuits, etc., may extend along at least part of the length of inner lumen (520), external to working channel (506).

As also shown in FIG. 5, proximal tendon assemblies (530) extend through inner lumen (520), external to working channel (506), in the present example. In some versions, proximal tendon assemblies (530) are freely disposed within inner lumen (520). In some other versions, one or more features or techniques are used to maintain the positioning of proximal tendon assemblies (530) along the inner surface of inner shaft (522), such that proximal tendon assemblies (530) are fixedly secured within inner lumen (520). In the present example, elongate member (500) includes four proximal tendon assemblies (530) that are angularly spaced apart from each other equidistantly (i.e., by approximately 90 degrees). In some other versions, between one and three proximal tendon assemblies (530) are provided; while in other versions more than four proximal tendon assemblies (530) are provided. Each proximal tendon assembly (530) includes a housing (532) and a tendon (536) slidably disposed in a lumen (534) defined by housing (532). Housing (532) is configured to deform laterally yet not compress longitudinally. By way of example only, housing (532) may be configured as a coil pipe formed of round or square steel wire. As another example, housing (532) housing may include several wires wrapped in multiple adjacent helixes. Alternatively, housing (532) may take any other suitable form. In some variations, housing (532) includes a low-friction (e.g., polytetrafluoroethylene) lining within lumen (534).

Each tendon (536) has a distal end that is fixedly secured at or near distal end (504) of elongate member (500) to provide articulation of distal portion (512). In some other variations, one or more tendons (536) has a distal end that is fixedly secured at some position that is proximal to distal portion (512). By way of example only, each tendon (536) may comprise a pull wire, a drive band, a single-strand cable, a multi-strand cable, one or more metals, one or more fibers, and/or any other suitable component that is operable to communicate a pulling force along the length of elongate member (500), to thereby provide articulation of elongate member (500), without substantially stretching. Such tendons (536) may also be coupled with instrument coupling (11) of robotic surgical system (10), such that robotic surgical system (10) is operable to drive tendons (536) via instrument coupling (11).

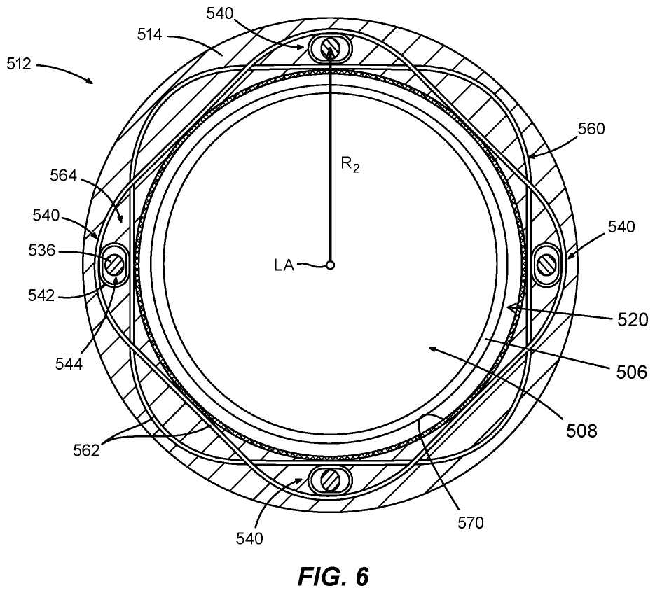

As shown in FIG. 6, distal portion (512) includes a body (514), a braid assembly (560), and an inner shaft (570). By way of example only, inner shaft (570) may include a flexible laser-cut steel hypotube, a braided structure, a link assembly, or any other suitable kind of structure. Inner shaft (570) provides a continuation of inner lumen (520) defined by inner shaft (522) and described above. Thus, working channel (506) and/or other components (e.g., electrical wires, etc.) disposed in inner lumen (520) in proximal portion (510) may freely continue through inner lumen (520) in distal portion (512). Braid assembly (560) includes a plurality of wire strands (562) that are wrapped to form an elongated braided structure that extends along the length of distal portion (512).

Distal portion (512) of the present example further includes distal tendon assemblies (540). As will be described in greater detail below, each distal tendon assembly (540) is associated with a corresponding proximal tendon assembly (530), by sharing a common tendon (536). Each distal tendon assembly (540) includes a housing (542) that defines a lumen (544), with a corresponding tendon (536) disposed in lumen (544). By way of example only, housing (542) may be configured as a tubular body that is embedded within the wall of braid assembly (560) (e.g., woven between inner and outer wire strands (562) of braid assembly (560)). Alternatively, housing (542) may take any other suitable form. In some variations, housing (542) includes a low-friction (e.g., polytetrafluoroethylene, polyimide, etc.) lining within lumen (544).

As shown in FIG. 6, distal tendon assemblies (540) are woven in with braid assembly (560), such that each distal tendon assembly (540) is disposed in a space (564) defined by wire strands (562). In some other versions, each distal tendon assembly (540) is radially interposed between braid assembly (560) and inner shaft (570). In either scenario, each distal tendon assembly (540) may extend along a path that is parallel with the central longitudinal axis (LA) of distal portion (512). It should also be understood that positioning distal tendon assemblies (540) within braid assembly (560) or otherwise radially outside of inner shaft (570) may maximize the amount of space available within the portion of inner lumen (520) that extends through distal portion (512). This may allow the portion of inner lumen (520) that extends through distal portion (512) to more readily accommodate components associated with functionalities provided at distal end (504) (e.g., components of one or more cameras at distal end (504), mechanical components of a mechanically-actuated end effector at distal end (504), etc.).

Body (514) of the present example is formed about the exterior of braid assembly (560). By way of example only, body (514) may be formed about the exterior of braid assembly (560) through a reflow process and/or through any other suitable process. Body (514) may comprise a reflow material such as polyether block amide (PEBA) and/or any other suitable kind(s) of material(s). At least some of the material used to form the region of body (514) outside of braid assembly (560) may also reach the region between braid assembly (560) and inner shaft (570), as shown in FIG. 6. In scenarios where distal tendon assemblies (540) would otherwise tend to exert outwardly directed forces on body (514) (e.g., during bending of elongate member (500), particularly during driven articulation), braid assembly (560) may effectively absorb such forces and thereby shield body (514) from any damage that might otherwise be caused to body (514) by distal tendon assemblies (540). With distal tendon assemblies (540) being woven between inner and outer wire strands (562) of braid assembly (560), braid assembly (560) may also maintain the angular position of distal tendon assemblies (540) about the central longitudinal axis (LA).

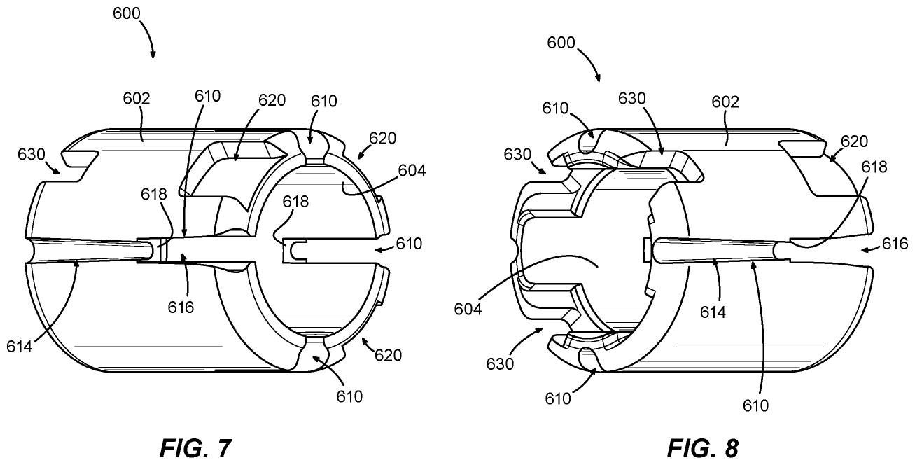

Coupler (600) is longitudinally interposed between proximal portion (510) and distal portion (512). As shown in FIGS. 7-8, coupler (600) includes a hollow body (602) that defines a plurality of longitudinally extending channels (610), an array of distal recesses (630), and an array of proximal recesses (620). The hollow configuration of body (602) allows working channel to pass continuously from proximal portion (510) to distal portion (512) via coupler (600). Body (602) is formed of a rigid material (e.g., molded plastic, etc.) as a single, monolithic piece. In some other versions, hollow body (602) is formed as an assembly of components (e.g., stacked discs, etc.).

Channels (610) are angularly spaced apart from each other equidistantly (i.e., by approximately 90 degrees) and extend along the full length of body (602). Each channel (610) includes a distal portion (614) and a proximal portion (616). Each proximal portion (616) opens through inner surface (604) of body (602); while each distal portion (614) does not open through inner surface (604) of body (602). A proximally facing shoulder surface (618) is positioned at the transition from proximal portion (616) to distal portion (614).

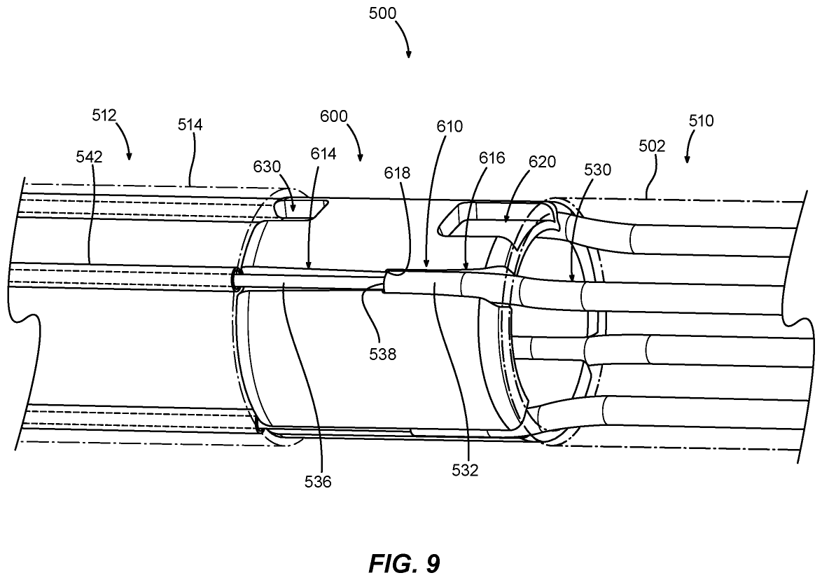

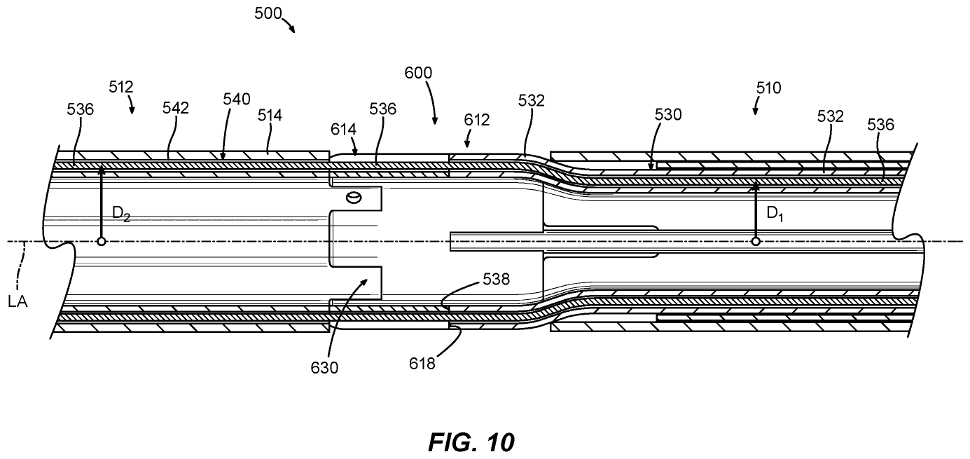

As shown in FIGS. 9-10 (in which working channel (506) is omitted for clarity), coupler (600) is configured to abut the distal end of proximal portion (510) and the proximal end of distal portion (512). Since body (602) is rigid, coupler (600) prevents longitudinal movement of the proximal end of distal portion (512) relative to the distal end of proximal portion (510). In other words, coupler (600) provides a mechanical ground between the distal end of proximal portion (510) and the proximal end of distal portion (512). As is also shown in FIGS. 9-10, proximal portion (616) of each channel (610) is configured to receive a corresponding housing (532) of each proximal tendon assembly (530); while distal portion (614) of each channel (610) is configured to receive a corresponding tendon (536). With housing (532) fitted in proximal portion (616) of channel (610), distal end (538) of housing (532) abuts proximally facing shoulder surface (618). This engagement between distal end (538) of housing (532) and proximally facing shoulder surface (618) provides a mechanical ground between housing (532), coupler (600), distal end of proximal portion (510), and the proximal end of distal portion (512).

As shown in FIG. 5, each tendon (536) is positioned at a first radial distance (R₁) from the central longitudinal axis (LA) along proximal portion (510). As shown in FIG. 6, each tendon (536) is positioned at a second radial distance (R₂) from the central longitudinal axis (LA) along distal portion (512). Second radial distance (R₂) is larger than first radial distance (R₁). Thus, to effectively transition tendons (536) from proximal portion (510) to distal portion (512), channels (610) guide tendons radially outwardly from the first radial distance (R₁) to the second radial distance (R₂). This is best seen in FIG. 10. As noted above, each proximal portion (616) of each channel (610) opens through inner surface (604) of body (602). This allows the distal region of each proximal tendon assembly (530) to bend away from the central longitudinal axis (LA) and pass through the proximal portion of coupler (600), thereby allowing distal end (538) of housing (532) to engage proximally facing shoulder surface (618); and tendon (536) to pass into distal portion (614) of channel (610).

Referring back to FIGS. 7-8, coupler (600) of the present example includes three distal recesses (630), which are angularly spaced apart from each other. Similarly, coupler (600) of the present example includes three proximal recesses (620), which are angularly spaced apart from each other. Any other suitable number of recesses (620, 630) may be provided, and recesses (620, 630) may be provided in any other suitable arrangement. Distal recesses (630) are configured to engage with complementary features (e.g., tabs) at the proximal end of distal portion (512); and proximal recesses (620) with complementary features (e.g., tabs) at the distal end of proximal portion (510). In the present example, recesses (620, 630) and the complementary features of portions (510, 512) are positioned asymmetrically about the central longitudinal axis (LA), such that two of recesses (620, 630) are angularly separated from each other by 180 degrees in one angular region of coupler (600); and recesses (620, 630) are angularly separated from each other by 90 degrees in the other angular region of coupler (600). This relationship may provide a consistent, predefined angular positioning between coupler (600) and distal portion (512) about the central longitudinal axis (LA). Similarly, proximal recesses (520) are configured to engage with complementary features at the distal end of proximal portion (510), thereby providing a consistent, predefined angular positioning between coupler (600) and proximal portion (510) about the central longitudinal axis (LA). It should therefore be understood that coupler (600) may serve as a poke-yoke feature ensuring that proximal portion (510) and distal portion (512) are appropriately aligned with each other angularly about the central longitudinal axis (LA).

While three recesses (620) are provided in this example, other variations may include just one recess (620) or more than two recesses (620). The number of complementary features (e.g., tabs, etc.) at the proximal end of distal portion (512) may vary accordingly. Similarly, while three recesses (630) are provided in this example, other variations may include just one recess (630) or more than two recesses (630). The number of complementary features (e.g., tabs, etc.) at the distal end of proximal portion (510) may vary accordingly.

As noted above, body (514) may be formed about the exterior of braid assembly (560) through a reflow process using a reflow material such as polyether block amide (PEBA) and/or any other suitable kind(s) of material(s). It should also be understood that the entire length of elongate member (500) may have an outer layer formed using a reflow material. Such an outer layer may extend continuously along both portions (510, 512) and coupler (600), thereby providing a smooth, continuous outer surface along the length of elongate member (500). In some such versions, the same reflow material (and process) that is used to form body (514) may be used to form such an outer layer that extends continuously along both portions (510, 512) and coupler (600). Alternatively, any other suitable kind of component (e.g., wrap, jacket, etc.), material, and process (e.g., heat shrink, etc.) may be used to form an outer layer that extends continuously along both portions (510, 512) and coupler (600).

While the examples described above include just two portions (510, 512) that are joined by a single coupler (600), other variations may include more than two portions that are joined by two or more couplers. For instance, some variations may include an elongate member with a proximal portion joined to a medial portion via a first coupler; and a distal portion joined to the medial portion via a second coupler. In some such variations, the proximal portion may provide flexibility but no articulation; the medial portion may provide articulation along only one plane; and the distal portion may provide articulation along two orthogonal planes independently of the articulation of the medial portion. As another example of a variation, the proximal portion may provide flexibility but no articulation; the medial portion may provide articulation along two orthogonal planes; and the distal portion may provide articulation along two orthogonal planes independently of the articulation of the medial portion. Still other variations will be apparent to those skilled in the art in view of the teachings herein. In any of these alternative scenarios, couplers like coupler (600) may facilitate the traversal of tendons (536) and other longitudinally extending components continuously across such different portions having different architectures and functionalities.

While channels (610) of coupler (600) in the present example are all parallel to the central longitudinal axis (LA) in this example, channels (610) may be oriented differently in other variations. For instance, in some variations, channels (610) may have helical orientations about the central longitudinal axis (LA). Such helical orientations of channels (610) may angularly reposition a tendon (536) or other longitudinally extending component, from a first angular position about the central longitudinal axis (LA) to a second angular position about the central longitudinal axis (LA), as the tendon (536) or other longitudinally extending component transitions from proximal portion (510) to distal portion (512). As noted above, some elongate members may include multiple couplers, such that this angular re-positioning may be provided multiple times along the length of the elongate member, with each of the couplers providing their own repositioning of tendons (536) or other longitudinally extending components.

While the examples described above provide tendons (536) within lumens (534, 544) of housings (532, 542), other variations may provide other kinds of components within lumens (534, 544) of housings (532, 542). Examples of such other components include, but are not limited to, electrical wires, irrigation channels, optical fibers, etc.

### III. EXAMPLES OF COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) a proximal elongate portion, the proximal elongate portion being flexible; (b) a distal elongate portion, the distal elongate portion being flexible, the proximal and distal elongate portions sharing a central longitudinal axis; (c) a coupler longitudinally interposed between the proximal elongate portion and the distal elongate portion, the coupler including a first channel; (d) a first proximal tendon assembly extending through the proximal elongate portion, the first proximal tendon assembly including a proximal portion of a first tendon positioned at a first radial distance from the central longitudinal axis; and (e) a first distal tendon assembly extending through the distal elongate portion, the first distal tendon assembly including a distal portion of the first tendon positioned at a second radial distance from the central longitudinal axis; the first tendon passing through the first channel of the coupler; the first channel of the coupler accommodating repositioning of the first tendon from the first radial distance to the second radial distance as the first tendon passes through the first channel of the coupler.

### Example 2

The apparatus of Example 1, the coupler being configured to provide a mechanical ground between a distal end of the first elongate portion and a proximal end of the second elongate portion.

### Example 3

The apparatus of Example 2, the coupler abutting the distal end of the first elongate portion, the coupler further abutting the proximal end of the second elongate portion.

### Example 4

The apparatus of any of Examples 1 through 3, the first tendon assembly being operable to drive articulation of the distal elongate portion.

### Example 5

The apparatus of any of Examples 1 through 4, the first proximal tendon assembly comprising a housing, the proximal portion of the first tendon being slidably disposed in the housing of the first proximal tendon assembly.

### Example 6

The apparatus of Example 5, the housing having a distal end, the distal end of the housing abutting a surface of the coupler.

### Example 7

The apparatus of any of Examples 5 through 6, the housing comprising a coil pipe.

### Example 8

The apparatus of any of Examples 1 through 7, the proximal elongate portion defining a proximal portion of an inner lumen.

### Example 9

The apparatus of Example 8, the first proximal tendon assembly being positioned in the inner lumen.

### Example 10

The apparatus of Example 9, the first proximal tendon assembly being movable within the inner lumen.

### Example 11

The apparatus of any of Examples 8 through 10, the distal elongate portion defining a distal portion of the inner lumen.

### Example 12

The apparatus of Example 11, the inner lumen extending continuously from the proximal elongate portion to the distal elongate portion via the coupler.

### Example 13

The apparatus of any of Examples 11 through 12, the first distal tendon assembly being positioned radially outwardly from the inner lumen.

### Example 14

The apparatus of Example 13, the distal elongate portion including an inner shaft, the inner shaft defining the distal portion of the inner lumen, the first distal tendon assembly being positioned radially outwardly from the inner shaft.

### Example 15

The apparatus of Example 14, the inner shaft comprising a laser cut hypotube.

### Example 16

The apparatus of any one or more of Examples 1 through 15, the distal elongate portion including a braid assembly.

### Example 17

The apparatus of Example 16, the first distal tendon assembly being interposed between strands forming the braid assembly.

### Example 18

The apparatus of any one or more of Examples 1 through 17, the first distal tendon assembly comprising a housing, the distal portion of the first tendon being slidably disposed in the housing of the first distal tendon assembly.

### Example 19

The apparatus of any of Examples 1 through 18, the coupler including a second channel, the apparatus further comprising: (a) a second proximal tendon assembly extending through the proximal elongate portion, the second proximal tendon assembly including a proximal portion of a second tendon positioned at the first radial distance from the central longitudinal axis; and (b) a second distal tendon assembly extending through the distal elongate portion, the second distal tendon assembly extending including a distal portion of the second tendon positioned at the second radial distance from the central longitudinal axis; the second tendon passing through the second channel of the coupler; the second channel of the coupler accommodating repositioning of the second tendon from the first radial distance to the second radial distance as the second tendon passes through the second channel of the coupler.

### Example 20

The apparatus of Example 19, the first tendon being angularly offset from the second tendon about the central longitudinal axis by approximately 90 degrees.

### Example 21

The apparatus of Example 19, the first tendon being angularly offset from the second tendon about the central longitudinal axis by approximately 180 degrees.

### Example 22

The apparatus of any of Examples 1 through 21, the coupler including a distal set of angular alignment features, the distal set of angular alignment features being configured to mate with complementary features at a proximal end of the distal elongate portion to provide a predetermined angular alignment of the coupler relative to the distal elongate portion.

### Example 23

The apparatus of any of Examples 1 through 22, the coupler including a proximal set of angular alignment features, the proximal set of angular alignment features being configured to mate with complementary features at a distal end of the proximal elongate portion to provide a predetermined angular alignment of the coupler relative to the proximal elongate portion.

### Example 24

The apparatus of any of Examples 1 through 23, the distal elongate portion including one or more of the following: one or more cameras, one or more light sources, or one or more sensors.

### Example 25

The apparatus of any of Examples 1 through 24, the first channel extending along a path that is parallel with the central longitudinal axis.

### Example 26

An apparatus comprising: (a) a proximal elongate portion, the proximal elongate portion being flexible; (b) a distal elongate portion, the distal elongate portion being flexible, the proximal and distal elongate portions sharing a central longitudinal axis; (c) a coupler longitudinally interposed between the proximal elongate portion and the distal elongate portion, the coupler including a plurality of channels; (d) a plurality of proximal tendon assemblies extending through the proximal elongate portion, each proximal tendon assembly of the plurality of proximal tendon assemblies including a proximal portion of a respective tendon positioned at a first radial distance from the central longitudinal axis; and (e) a plurality of distal tendon assemblies extending through the distal elongate portion, each distal tendon assembly of the plurality of distal tendon assemblies including a distal portion of a respective tendon positioned at a second radial distance from the central longitudinal axis; each tendon passing through a respective channel of the plurality of channels of the coupler; each channel of the coupler accommodating repositioning of a respective tendon from the first radial distance to the second radial distance as the respective tendon passes through the first channel of the coupler.

### Example 27

A method comprising: (a) positioning a plurality of proximal tendon assemblies along a proximal elongate portion, the proximal tendon assemblies being positioned at a first radial distance from a central longitudinal axis defined by the proximal elongate portion; (b) positioning tendons of the plurality of proximal tendon assemblies along respective channels of a coupler, the coupler repositioning the tendons from the first radial distance to a second radial distance from the central longitudinal axis; and (c) positioning the tendons along a distal elongate portion, the tendons being positioned at the second radial distance along the distal elongate portion, the coupler being longitudinally interposed between the proximal elongate portion and the distal elongate portion.

### Example 28

The method of Example 27, the proximal tendon assemblies further including a plurality of housings, each tendon being slidably disposed in a respective housing of the plurality of housings.

### Example 29

The method of Example 28, each housing having a distal end, the method further comprising abutting the distal end of each housing against a corresponding surface of the coupler, thereby mechanically grounding the housing relative to the coupler.

### Example 30

The method of any of Examples 27 through 29, the coupler including a set of angular alignment features, the method further comprising aligning the angular alignment features of the coupler with complementary features of the proximal elongate portion and the distal elongate portion to thereby provide a predetermined angular alignment between the coupler, the proximal elongate portion, and the distal elongate portion.

### IV. MISCELLANEOUS

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they may be designed to be used multiple times. Versions may in either or both cases, be reconditioned for reuse after at least one use. Reconditioning may include any combination of the steps of disassembly of the systems, instruments, and/or portions thereof, followed by cleaning or replacement of particular pieces, and subsequent reassembly. In particular, some versions of the systems, instruments, and/or portions thereof may be disassembled, and any number of the particular pieces or parts of the systems, instruments, and/or portions thereof may be selectively replaced or removed in any combination. Upon cleaning and/or replacement of particular parts, some versions of the systems, instruments, and/or portions thereof may be reassembled for subsequent use either at a reconditioning facility, or by an operator immediately prior to a procedure. Those skilled in the art will appreciate that reconditioning of systems, instruments, and/or portions thereof may utilize a variety of techniques for disassembly, cleaning/replacement, and reassembly. Use of such techniques, and the resulting reconditioned systems, instruments, and/or portions thereof, are all within the scope of the present application.

By way of example only, versions described herein may be sterilized before and/or after a procedure. In one sterilization technique, the systems, instruments, and/or portions thereof is placed in a closed and sealed container, such as a plastic or TYVEK bag. The container and system, instrument, and/or portion thereof may then be placed in a field of radiation that may penetrate the container, such as gamma radiation, x-rays, or high-energy electrons. The radiation may kill bacteria on the system, instrument, and/or portion thereof and in the container. The sterilized systems, instruments, and/or portions thereof may then be stored in the sterile container for later use. Systems, instruments, and/or portions thereof may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/we claim:

1. An apparatus comprising:
(a) a proximal elongate portion, the proximal elongate portion being flexible;
(b) a distal elongate portion, the distal elongate portion being flexible, the proximal and distal elongate portions sharing a central longitudinal axis;
(c) a coupler longitudinally interposed between the proximal elongate portion and the distal elongate portion, the coupler including a first channel;
(d) a first proximal tendon assembly extending through the proximal elongate portion, the first proximal tendon assembly including a proximal portion of a first tendon positioned at a first radial distance from the central longitudinal axis; and
(e) a first distal tendon assembly extending through the distal elongate portion, the first distal tendon assembly including a distal portion of the first tendon positioned at a second radial distance from the central longitudinal axis;
the first tendon passing through the first channel of the coupler;
the first channel of the coupler accommodating repositioning of the first tendon from the first radial distance to the second radial distance as the first tendon passes through the first channel of the coupler.

2. The apparatus of claim 1, the coupler being configured to provide a mechanical ground between a distal end of the first elongate portion and a proximal end of the second elongate portion.

3. The apparatus of claim 2, the coupler abutting the distal end of the first elongate portion, the coupler further abutting the proximal end of the second elongate portion.

4. The apparatus of claim 1, the first tendon assembly being operable to drive articulation of the distal elongate portion.

5. The apparatus of claim 1, the first proximal tendon assembly comprising a housing, the proximal portion of the first tendon being slidably disposed in the housing of the first proximal tendon assembly.

6. The apparatus of claim 5, the housing having a distal end, the distal end of the housing abutting a surface of the coupler.

7. The apparatus of claim 5, the housing comprising a coil pipe.

8. The apparatus of claim 1, the proximal elongate portion defining a proximal portion of an inner lumen.

9. The apparatus of claim 8, the first proximal tendon assembly being positioned in the inner lumen.

10. The apparatus of claim 9, the first proximal tendon assembly being movable within the inner lumen.

11. The apparatus of claim 8, the distal elongate portion defining a distal portion of the inner lumen.

12. The apparatus of claim 11, the inner lumen extending continuously from the proximal elongate portion to the distal elongate portion via the coupler.

13. The apparatus of claim 11, the first distal tendon assembly being positioned radially outwardly from the inner lumen.

14. The apparatus of claim 13, the distal elongate portion including an inner shaft, the inner shaft defining the distal portion of the inner lumen, the first distal tendon assembly being positioned radially outwardly from the inner shaft.

15. The apparatus of claim 1, the distal elongate portion including a braid assembly.

16. The apparatus of claim 1, the first distal tendon assembly comprising a housing, the distal portion of the first tendon being slidably disposed in the housing of the first distal tendon assembly.

17. The apparatus of claim 1, the coupler including a second channel, the apparatus further comprising:
(a) a second proximal tendon assembly extending through the proximal elongate portion, the second proximal tendon assembly including a proximal portion of a second tendon positioned at the first radial distance from the central longitudinal axis; and
(b) a second distal tendon assembly extending through the distal elongate portion, the second distal tendon assembly extending including a distal portion of the second tendon positioned at the second radial distance from the central longitudinal axis;
the second tendon passing through the second channel of the coupler;
the second channel of the coupler accommodating repositioning of the second tendon from the first radial distance to the second radial distance as the second tendon passes through the second channel of the coupler.

18. An apparatus comprising:
(a) a proximal elongate portion, the proximal elongate portion being flexible;
(b) a distal elongate portion, the distal elongate portion being flexible, the proximal and distal elongate portions sharing a central longitudinal axis;
(c) a coupler longitudinally interposed between the proximal elongate portion and the distal elongate portion, the coupler including a plurality of channels;
(d) a plurality of proximal tendon assemblies extending through the proximal elongate portion, each proximal tendon assembly of the plurality of proximal tendon assemblies including a proximal portion of a respective tendon positioned at a first radial distance from the central longitudinal axis; and
(e) a plurality of distal tendon assemblies extending through the distal elongate portion, each distal tendon assembly of the plurality of distal tendon assemblies including a distal portion of a respective tendon positioned at a second radial distance from the central longitudinal axis;
each tendon passing through a respective channel of the plurality of channels of the coupler;
each channel of the coupler accommodating repositioning of a respective tendon from the first radial distance to the second radial distance as the respective tendon passes through the first channel of the coupler.

19. A method comprising:
(a) positioning a plurality of proximal tendon assemblies along a proximal elongate portion, the proximal tendon assemblies being positioned at a first radial distance from a central longitudinal axis defined by the proximal elongate portion;
(b) positioning tendons of the plurality of proximal tendon assemblies along respective channels of a coupler, the coupler repositioning the tendons from the first radial distance to a second radial distance from the central longitudinal axis; and
(c) positioning the tendons along a distal elongate portion, the tendons being positioned at the second radial distance along the distal elongate portion, the coupler being longitudinally interposed between the proximal elongate portion and the distal elongate portion.

20. The method of claim 19, the proximal tendon assemblies further including a plurality of housings, each tendon being slidably disposed in a respective housing of the plurality of housings.
